(19) United States
(12) Patent Application Publication
(10) Pub. No.: US 2022/0395575 A1
(43) Pub. Date: Dec. 15, 2022

(54) COMBINATION THERAPY WITH PROTEIN KINASE B ACTIVATION INHIBITOR TO TREAT CANCER

(71) Applicant: H. LEE MOFFITT CANCER CENTER AND RESEARCH INSTITUTE, INC., Tampa, FL (US)

(72) Inventor: Said M. SEBTI, Glen Allen, VA (US)

(21) Appl. No.: 17/776,760

(22) PCT Filed: Nov. 16, 2020
(86) PCT No.: PCT/US2020/060670

§ 371 Date: May 13, 2022

Related U.S. Application Data
(60) Provisional application No. 62/935,392, filed on Nov. 14, 2019.

(51) Int. Cl. A61K 45/06; A61K 31/517; A61K 31/52; A61K 31/5377; A61K 31/7064; A61P 35/00

(52) U.S. Cl. CPC A61K 45/06; A61K 31/517; A61K 31/52; A61K 31/5377; A61K 31/7064; A61P 35/00

(57) ABSTRACT

Disclosed are synergistic compositions comprising a therapeutically effective synergistic amount of a protein kinase B (Akt) activation inhibitor and a second inhibitor selected from a checkpoint (CHK1) inhibitor, a multitargeted histone deacetylase/human epidermal growth factor receptor (HDAC/Her1/2) inhibitor, a phosphoinositide 3-kinase (PI3K) inhibitor, or a combination thereof. Methods of using the disclosed compositions for treating, preventing, reducing, and/or inhibiting a cancer comprising administering the same to a subject are also disclosed.

### BACKGROUND

Hyperactived Akt is observed in over 50% of human tumors and is the hallmark of many aggressive cancers (Altomare D A, Testa J R. Oncogene 2005; 24(50):7455-64). Akt is an AGC family serine/threonine kinase and a cardinal nodal point in the PI3K signaling pathway. It plays an important role in conferring resistance to apoptosis through phosphorylation and inactivation of substrates such as GSK3α/β, Bad, and FOXO3a (Testa J R, Bellacosa A. Proc. Natl. Acad. Sci. USA. 2001; 98(20):10983-5). Several small molecule Akt inhibitors are currently in clinical trials. However, there is still a need for improved strategies for treating human tumors. The compositions and methods disclosed herein address these and other needs.

### SUMMARY

In accordance with the purposes of the disclosed materials and methods, as embodied and broadly described herein, the disclosed subject matter, in one aspect, relates to compositions and methods of using the compositions. Disclosed are compositions comprising a therapeutically effective synergistic amount of a protein kinase B (Akt) activation inhibitor and a second inhibitor selected from a checkpoint (CHK1) inhibitor, a multitargeted histone deacetylase/human epidermal growth factor receptor (HDAC/Her1/2) inhibitor, a phosphoinositide 3-kinase (PI3K) inhibitor, or a combination thereof. The Akt activation inhibitor can include an inhibitor that inhibits Akt phosphorylation. For example, the Akt activation inhibitor can include triciribine phosphate (TCN-P).

In some embodiments, the composition includes the Akt activation inhibitor and the CHK1 inhibitor in a therapeutically effective synergistic amount. The CHK1 inhibitor can include an inhibitor that inhibits Chk1 phosphorylation. For example, the CHK1 inhibitor can include rabusertib (LY2603618).

In some embodiments, the composition includes the Akt activation inhibitor and the multitargeted HDAC/Her1/2 inhibitor in a therapeutically effective synergistic amount. Specific examples of multitargeted HDAC/Her1/2 inhibitor include CUDC-101.

In some embodiments, the composition includes the Akt activation inhibitor and the PI3K inhibitor in a therapeutically effective synergistic amount. The PI3K inhibitor can include an inhibitor that inhibits both delta and gamma isoforms of PI3K. For example, the PI3K inhibitor can include duvelisib (IPI-145).

Methods of using the disclosed compositions for treating, preventing, reducing, and/or inhibiting a cancer comprising administering the same to a subject are also disclosed. The cancer can be selected from breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma). In some examples, the cancer is leukemia. The cancer can be in a human.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

FIG. 1 shows graphical representations of CHK1 inhibitor LY2603618 synergizes with TCN-P to inhibit viability of HL60 human leukemic cells.

FIG. 2 shows graphical representations of CHK1 inhibitor LY2603618 synergizes with TCN-P to inhibit viability of THP1 human leukemic cells.

FIG. 3 shows graphical representations of CHK1 inhibitor LY2603618 synergizes with TCN-P to inhibit viability of THP1 human leukemic cells.

FIG. 4 shows graphical representations of CHK1 inhibitor LY2603618 synergizes with TCN-P to inhibit viability of U937 human leukemic cells.

FIG. 5 shows graphical representations of CHK1 inhibitor LY2603618 synergizes with TCN-P to inhibit viability of THP1, HL60 and U937 human leukemic cells.

FIG. 6 shows graphical representations of HDAC/Her1/2 inhibitor CUDC-101 synergizes with TCN-P to inhibit viability of HL60 human leukemic cells.

FIG. 7 shows graphical representations of HDAC/Her1/2 inhibitor CUDC-101 synergizes with TCN-P to inhibit viability of THP1 human leukemic cells.

FIG. 8 shows graphical representations of HDAC/Her1/2 inhibitor CUDC-101 synergizes with TCN-P to inhibit viability of U937 human leukemic cells.

FIG. 9 shows graphical representations of HDAC/Her1/2 inhibitor CUDC-101 synergizes with TCN-P to inhibit viability of THP1, U937 and HL60 human leukemic cells.

FIG. 10 shows graphical representations of PI3K inhibitor IPI-145 synergizes with TCN-P to inhibit viability of HL60 human leukemic cells.

FIG. 11 shows graphical representations of PI3K inhibitor IPI-145 synergizes with TCN-P to inhibit viability of THP1 human leukemic cells.

FIG. 12 shows graphical representations of PI3K inhibitor IPI-145 synergizes with TCN-P to inhibit viability of U937 human leukemic cells.

FIG. 13 shows graphical representations of PI3K inhibitor IPI-145 synergizes with TCN-P to inhibit viability of U937, THP1 and HL60 human leukemic cells.

### DETAILED DESCRIPTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

By “reduce” or other forms of the word, such as “reducing” or “reduction,” is meant lowering of an event or characteristic (e.g., tumor growth, metastasis). It is understood that this is typically in relation to some standard or expected value, in other words it is relative, but that it is not always necessary for the standard or relative value to be referred to. For example, “reduces tumor growth” means decreasing the amount of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as tumor growth or metastasis), diminishment of extent of cancer, stabilized (i.e., not worsening) state of cancer, preventing or delaying spread (e.g., metastasis) of the cancer, delaying occurrence or recurrence of cancer, delay or slowing of cancer progression, amelioration of the cancer state, and remission (whether partial or total).

The term “patient” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more doses. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

Effective amounts of a compound or composition described herein for treating a mammalian subject can include about 0.01 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

Reference will now be made in detail to specific aspects of the disclosed materials, compounds, compositions, articles, and methods, examples of which are illustrated in the accompanying Examples.

### Compositions

Disclosed are pharmaceutical compositions comprising a) a protein kinase B (Akt) activation inhibitor and b) a second inhibitor selected from a checkpoint (CHK1) inhibitor, a multitargeted histone deacetylase/human epidermal growth factor receptor (HDAC/Her1/2) inhibitor, a phosphoinositide 3-kinase (PI3K) inhibitor, or a combination thereof. In some embodiments, the pharmaceutical compositions comprise a) a protein kinase B (Akt) activation inhibitor and b) a second inhibitor selected from a checkpoint (CHK1) inhibitor, a multitargeted histone deacetylase/human epidermal growth factor receptor (HDAC/Her1/2) inhibitor, or a combination thereof. In some embodiments, the pharmaceutical compositions comprise a) a protein kinase B (Akt) activation inhibitor and b) a second inhibitor selected from a checkpoint (CHK1) inhibitor, a phosphoinositide 3-kinase (PI3K) inhibitor, or a combination thereof. In some embodiments, the pharmaceutical compositions comprise a) a protein kinase B (Akt) activation inhibitor and b) a second inhibitor selected from a multitargeted histone deacetylase/human epidermal growth factor receptor (HDAC/Her1/2) inhibitor, a phosphoinositide 3-kinase (PI3K) inhibitor, or a combination thereof.

The Akt activation inhibitor can include an inhibitor that inhibits Akt phosphorylation. For example, the Akt activation inhibitor can include triciribine phosphate (TCN-P). Other inhibitors of Akt are detailed in Nitulescu G. et al., (Int J Oncol. 2016 March; 48(3): 869-885) and include ATP-competitive inhibitors (which can be orthosteric inhibitors targeting the ATP-binding pocket of the protein kinase B (Akt)); isoquinoline-5-sulfonamides; azepane derivatives; aminofurazans (e.g., GSK690693); heterocyclic rings (e.g., 7-azaindole, 6-phenylpurine derivatives, pyrrolo[2,3-d]pyrimidine derivatives, CCT128930, 3-aminopyrrolidine, anilinotriazole derivatives, spiroindoline derivatives, AZD5363, ipatasertib (GDC-0068, RG7440), A-674563, A-443654); phenylpyrazole derivatives (e.g., AT7867, AT13148); thiophenecarboxamide derivatives (e.g., afuresertib (GSK2110183), 2-pyrimidyl-5-amidothiophene derivative (DC120), and uprosertib (GSK2141795); allosteric inhibitors; 2,3-diphenylquinoxaline analogues (e.g., 2,3-diphenylquinoxaline derivatives, triazolo[3,4-f][1,6]naphthyridin-3(2H)-one derivative (MK-2206)); alkylphospholipids (e.g., edelfosine (1-O-octadecyl-2-O-methyl-rac-glycero-3-phosphocholine, ET-18-OCH₃), ilmofosine (BM 41.440), miltefosine (hexadecylphosphocholine, HePC), perifosine (D-21266), erucylphosphocholine (ErPC), and erufosine (ErPC3, erucylphosphohomocholine); indole-3-carbinol analogues (e.g., indole-3-carbinol, 3-chloroacetylindole, diindolylmethane, diethyl 6-methoxy-5,7-dihydroindolo [2,3-b]carbazole-2,10-dicarboxylate (SR13668), and OSU-A9); sulfonamide derivatives (e.g., PH-316, PHT-427); thiourea derivatives (e.g., PIT-1, PIT-2, DM-PIT-1, N-[(1-methyl-1H-pyrazol-4-yl)carbonyl]-N′-(3-bromophenyl)-thiourea); purine derivatives (e.g., triciribine (TCN, NSC 154020), triciribine mono-phosphate active analogue (TCN-P), 4-amino-pyrido[2,3-d]pyrimidine derivative API-1, 3-phenyl-3H-imidazo[4,5-b]pyridine derivatives, ARQ 092); other structural derivatives (e.g., BAY 1125976, 3-methyl-xanthine, quinoline-4-carboxamide and 2-[4-(cyclohexa-1,3-dien-1-yl)-1H-pyrazol-3-yl]phenol, 3-oxo-tirucallic acid, 3α- and 3β-acetoxy-tirucallic acids, acetoxy-tirucallic acid); and irreversible inhibitors (e.g., natural products, antibiotics, lactoquinomycin, frenolicin B, kalafungin, medermycin, Boc-Phe-vinyl ketone, 4-hydroxynonenal (4-HNE), 1,6-naphthyridinone derivatives, and imidazo-1,2-pyridine derivative).

In some embodiments, the composition includes the Akt activation inhibitor and the CHK1 inhibitor in a therapeutically effective synergistic amount. The CHK1 inhibitor can include an inhibitor that inhibits Chk1 phosphorylation. For example, the CHK1 inhibitor can include rabusertib (LY2603618). Other CHK1 inhibitors are detailed in Prudhomme M., (Recent Patents on Anti-Cancer Drug Discovery, 2006, Vol. 1, No. 1 61) and include SCH900776, PF477736, CCT245737, CHIR-124, CCT244747, PD 407824, GDC-0575, SB-218078, Chk1-IN-5, prexasertib, AZD-7762, CCT241533, MRT00033659, MK-8776 (SCH900776), UCN-01 (7-hydroxystaurosporine); staurosporine, AZD7762, PF-0477736, LY2606368, SB-218078, hymenialdisine, debromohymenialdisine, 13-hydroxy-15-oxozoapatlin, PD0166285, granulatimide, isogranulatimide, and oxindole and pyrimidine Chk1 inhibitors.

In some embodiments, the composition includes the Akt activation inhibitor and the multitargeted HDAC/Her1/2 inhibitor in a therapeutically effective synergistic amount. Specific examples of multitargeted HDAC/Her1/2 inhibitor include CUDC-101. Other HDAC/Her1/2 inhibitors are detailed in Zhou N. et al., (Drug Discoveries & Therapeutics. 2015; 9(3):147-155) and include N-aryl saliylamides and lapatinib.

In some embodiments, the composition includes the Akt activation inhibitor and the PI3K inhibitor in a therapeutically effective synergistic amount. The PI3K inhibitor can include an inhibitor that inhibits both delta and gamma isoforms of PI3K. For example, the PI3K inhibitor can include duvelisib (IPI-145). Other PI3K inhibitors are detailed in Verret B. et al., (Annals of Oncology 30 (Supplement 10): x12-x20, 2019) and include buparlisib, alpelisib, taselisib, TAK-117, GDC-0077 (RG6114), HH-CYH33, BAY1082439, ON146040, zydelig (idelalisib), aliqopa (copanlisib), TGR-1202 (umbralisib), CDZ173 (lenioilisib), INCB050465, ACP-319, nemiralisib, buparlisib, GDC-0084, GSK2636771, KA2237, GSK2292767, HMPL-689, tenalisib, and IPI-549, PA799.

In some embodiments, (a) protein kinase B (Akt) activation inhibitor and b) second inhibitor can be used in an amount sufficient to induce a synergistic therapeutic effect while still showing good pharmaceutical compatibility (i.e., their use in a subject does not result in increased adverse effects to the subject when compared to the individual application of the therapeutic compounds (a) or (b)). In some embodiments, the joint action of the a) protein kinase B (Akt) activation inhibitor and b) the second inhibitor can result in enhanced activity against cancer (via synergism), even at therapeutic doses below those typically used for the therapeutic agent to have a therapeutic effect on its own. In some embodiments, the compositions and methods disclosed herein can, based on the individual components, be used at lower therapeutic doses to achieve a therapeutic effect comparable to the effect produced by the individual components at normal therapeutic doses. In some embodiments, the compositions and methods disclosed herein provide improved efficacy on tumors (i.e., they effect treatment more quickly and/or exhibit greater inhibition compared with application of the individual therapeutic agent).

In some embodiments, the observed anticancer effect is at least 1%, at least 2%, at least 3%, at least 4%, at least 5%, at least 10%, at least 15%, at least 20%, at least 25%, at least 27%, at least 30%, at least 35%, at least 40%, at least 45%, at least 50%, at least 55%, at least 60%, at least 65%, at least 70%, at least 75%, at least 80%, at least 85%, or at least 90% greater than the expected effect (e.g., an observed effect of 96% would be 4% greater than an expected effect of 92%).

The Akt activation inhibitor (e.g., triciribine phosphate) can be present in an amount sufficient to induce a therapeutic effect when administered to the subject. In some embodiments, the Akt activation inhibitor (e.g., triciribine phosphate) can be administered to the subject in an amount of 0.1 mg/kg grams or greater of active ingredient per body weight (e.g., 0.2 mg/kg or greater, 0.5 mg/kg or greater, 0.8 mg/kg or greater, 1 mg/kg or greater, 1.2 mg/kg or greater, 1.5 mg/kg or greater, 2 mg/kg or greater, 2.5 mg/kg or greater, 3 mg/kg or greater, 4 mg/kg or greater, 5 mg/kg or greater, 8 mg/kg or greater, 10 mg/kg or greater, 15 mg/kg or greater, 20 mg/kg or greater, 25 mg/kg or greater, 30 mg/kg or greater, 35 mg/kg or greater, 40 mg/kg or greater, 45 mg/kg or greater, 50 mg/kg or greater, 55 mg/kg or greater, 60 mg/kg or greater, 65 mg/kg or greater, 70 mg/kg or greater, 75 mg/kg or greater, 80 mg/kg or greater, 85 mg/kg or greater, 90 mg/kg or greater, 95 mg/kg or greater, 100 mg/kg or greater, 120 mg/kg or greater, 150 mg/kg or greater, 170 mg/kg or greater, 200 mg/kg or greater, 250 mg/kg or greater, 270 mg/kg or greater, 300 mg/kg or greater, 350 mg/kg or greater, 400 mg/kg or greater, 450 mg/kg or greater, 500 mg/kg or greater, 550 mg/kg or greater, 600 mg/kg or greater, 700 mg/kg or greater, 750 mg/kg or greater, 700 mg/kg or greater, 850 mg/kg or greater, 900 mg/kg or greater, 950 mg/kg or greater, or 1000 mg/kg or greater) per body weight.

In some embodiments, the Akt activation inhibitor (e.g., triciribine phosphate) can be administered to the subject in an amount of 1000 mg/kg grams or less of active ingredient per body weight (e.g., 950 mg/kg or less, 900 mg/kg or less, 850 mg/kg or less, 800 mg/kg or less, 750 mg/kg or less, 700 mg/kg or less, 650 mg/kg or less, 600 mg/kg or less, 550 mg/kg or less, 500 mg/kg or less, 450 mg/kg or less, 400 mg/kg or less, 350 mg/kg or less, 300 mg/kg or less, 250 mg/kg or less, 200 mg/kg or less, 150 mg/kg or less, 125 mg/kg or less, 120 mg/kg or less, 115 mg/kg or less, 110 mg/kg or less, 105 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 8 mg/kg or less, 6 mg/kg or less, or 5 mg/kg or less) per body weight.

The Akt activation inhibitor can be administered to the subject in an amount ranging from any of the minimum values described above to any of the maximum values described above. In some embodiments, the Akt activation inhibitor can be administered to the subject in an amount of from 0.01-1000 mg/kg (e.g., from 0.1-500 mg/kg, from 0.5-100 mg/kg, from 0.1-50 mg/kg, from 1-40 mg/kg, from 5-75 mg/kg, from 5-40 mg/kg, from 5-20 mg/kg, or from 2-20 mg/kg). In some embodiments, the Akt activation inhibitor is administered in an amount from 0.1-100 mg/kg.

The CHK1 inhibitor (e.g., rabusertib) can be present in an amount sufficient to induce a therapeutic effect when administered to the subject. In some embodiments, the CHK1 inhibitor (e.g., rabusertib) can be administered to the subject in an amount of 0.1 mg/kg grams or greater of active ingredient per body weight (e.g., 0.2 mg/kg or greater, 0.5 mg/kg or greater, 0.8 mg/kg or greater, 1 mg/kg or greater, 1.2 mg/kg or greater, 1.5 mg/kg or greater, 2 mg/kg or greater, 2.5 mg/kg or greater, 3 mg/kg or greater, 4 mg/kg or greater, 5 mg/kg or greater, 8 mg/kg or greater, 10 mg/kg or greater, 15 mg/kg or greater, 20 mg/kg or greater, 25 mg/kg or greater, 30 mg/kg or greater, 35 mg/kg or greater, 40 mg/kg or greater, 45 mg/kg or greater, 50 mg/kg or greater, 55 mg/kg or greater, 60 mg/kg or greater, 65 mg/kg or greater, 70 mg/kg or greater, 75 mg/kg or greater, 80 mg/kg or greater, 85 mg/kg or greater, 90 mg/kg or greater, 95 mg/kg or greater, 100 mg/kg or greater, 120 mg/kg or greater, 150 mg/kg or greater, 170 mg/kg or greater, 200 mg/kg or greater, 250 mg/kg or greater, 270 mg/kg or greater, 300 mg/kg or greater, 350 mg/kg or greater, 400 mg/kg or greater, 450 mg/kg or greater, 500 mg/kg or greater, 550 mg/kg or greater, 600 mg/kg or greater, 700 mg/kg or greater, 750 mg/kg or greater, 700 mg/kg or greater, 850 mg/kg or greater, 900 mg/kg or greater, 950 mg/kg or greater, or 1000 mg/kg or greater) per body weight.

In some embodiments, the CHK1 inhibitor (e.g., rabusertib) can be administered to the subject in an amount of 1000 mg/kg grams or less of active ingredient per body weight (e.g., 950 mg/kg or less, 900 mg/kg or less, 850 mg/kg or less, 800 mg/kg or less, 750 mg/kg or less, 700 mg/kg or less, 650 mg/kg or less, 600 mg/kg or less, 550 mg/kg or less, 500 mg/kg or less, 450 mg/kg or less, 400 mg/kg or less, 350 mg/kg or less, 300 mg/kg or less, 250 mg/kg or less, 200 mg/kg or less, 150 mg/kg or less, 125 mg/kg or less, 120 mg/kg or less, 115 mg/kg or less, 110 mg/kg or less, 105 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 8 mg/kg or less, 6 mg/kg or less, or 5 mg/kg or less) per body weight.

The CHK1 inhibitor (e.g., rabusertib) can be administered to the subject in an amount ranging from any of the minimum values described above to any of the maximum values described above. In some embodiments, the CHK1 inhibitor can be administered to the subject in an amount of from 0.01-1000 mg/kg (e.g., from 0.1-500 mg/kg, from 0.5-100 mg/kg, from 0.1-50 mg/kg, from 1-40 mg/kg, from 5-75 mg/kg, from 5-40 mg/kg, from 5-20 mg/kg, or from 2-20 mg/kg). In some embodiments, the CHK1 inhibitor is administered in an amount from 0.1-100 mg/kg.

The multitargeted HDAC/Her1/2 inhibitor (e.g., CUDC-101) can be present in an amount sufficient to induce a therapeutic effect when administered to the subject. In some embodiments, the multitargeted HDAC/Her1/2 inhibitor (e.g., CUDC-101) can be administered to the subject in an amount of 0.1 mg/kg grams or greater of active ingredient per body weight (e.g., 0.2 mg/kg or greater, 0.5 mg/kg or greater, 0.8 mg/kg or greater, 1 mg/kg or greater, 1.2 mg/kg or greater, 1.5 mg/kg or greater, 2 mg/kg or greater, 2.5 mg/kg or greater, 3 mg/kg or greater, 4 mg/kg or greater, 5 mg/kg or greater, 8 mg/kg or greater, 10 mg/kg or greater, 15 mg/kg or greater, 20 mg/kg or greater, 25 mg/kg or greater, 30 mg/kg or greater, 35 mg/kg or greater, 40 mg/kg or greater, 45 mg/kg or greater, 50 mg/kg or greater, 55 mg/kg or greater, 60 mg/kg or greater, 65 mg/kg or greater, 70 mg/kg or greater, 75 mg/kg or greater, 80 mg/kg or greater, 85 mg/kg or greater, 90 mg/kg or greater, 95 mg/kg or greater, 100 mg/kg or greater, 120 mg/kg or greater, 150 mg/kg or greater, 170 mg/kg or greater, 200 mg/kg or greater, 250 mg/kg or greater, 270 mg/kg or greater, 300 mg/kg or greater, 350 mg/kg or greater, 400 mg/kg or greater, 450 mg/kg or greater, 500 mg/kg or greater, 550 mg/kg or greater, 600 mg/kg or greater, 700 mg/kg or greater, 750 mg/kg or greater, 700 mg/kg or greater, 850 mg/kg or greater, 900 mg/kg or greater, 950 mg/kg or greater, or 1000 mg/kg or greater) per body weight.

In some embodiments, the multitargeted HDAC/Her1/2 inhibitor (e.g., CUDC-101) can be administered to the subject in an amount of 1000 mg/kg grams or less of active ingredient per body weight (e.g., 950 mg/kg or less, 900 mg/kg or less, 850 mg/kg or less, 800 mg/kg or less, 750 mg/kg or less, 700 mg/kg or less, 650 mg/kg or less, 600 mg/kg or less, 550 mg/kg or less, 500 mg/kg or less, 450 mg/kg or less, 400 mg/kg or less, 350 mg/kg or less, 300 mg/kg or less, 250 mg/kg or less, 200 mg/kg or less, 150 mg/kg or less, 125 mg/kg or less, 120 mg/kg or less, 115 mg/kg or less, 110 mg/kg or less, 105 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 8 mg/kg or less, 6 mg/kg or less, or 5 mg/kg or less) per body weight.

The multitargeted HDAC/Her1/2 inhibitor (e.g., CUDC-101) can be administered to the subject in an amount ranging from any of the minimum values described above to any of the maximum values described above. In some embodiments, the multitargeted HDAC/Her1/2 inhibitor can be administered to the subject in an amount of from 0.01-1000 mg/kg (e.g., from 0.1-500 mg/kg, from 0.5-100 mg/kg, from 0.1-50 mg/kg, from 1-40 mg/kg, from 5-75 mg/kg, from 5-40 mg/kg, from 5-20 mg/kg, or from 2-20 mg/kg). In some embodiments, the multitargeted HDAC/Her1/2 inhibitor is administered in an amount from 0.1-100 mg/kg.

The PI3K inhibitor (e.g., duvelisib) can be present in an amount sufficient to induce a therapeutic effect when administered to the subject. In some embodiments, the PI3K inhibitor (e.g., duvelisib) can be administered to the subject in an amount of 0.1 mg/kg grams or greater of active ingredient per body weight (e.g., 0.2 mg/kg or greater, 0.5 mg/kg or greater, 0.8 mg/kg or greater, 1 mg/kg or greater, 1.2 mg/kg or greater, 1.5 mg/kg or greater, 2 mg/kg or greater, 2.5 mg/kg or greater, 3 mg/kg or greater, 4 mg/kg or greater, 5 mg/kg or greater, 8 mg/kg or greater, 10 mg/kg or greater, 15 mg/kg or greater, 20 mg/kg or greater, 25 mg/kg or greater, 30 mg/kg or greater, 35 mg/kg or greater, 40 mg/kg or greater, 45 mg/kg or greater, 50 mg/kg or greater, 55 mg/kg or greater, 60 mg/kg or greater, 65 mg/kg or greater, 70 mg/kg or greater, 75 mg/kg or greater, 80 mg/kg or greater, 85 mg/kg or greater, 90 mg/kg or greater, 95 mg/kg or greater, 100 mg/kg or greater, 120 mg/kg or greater, 150 mg/kg or greater, 170 mg/kg or greater, 200 mg/kg or greater, 250 mg/kg or greater, 270 mg/kg or greater, 300 mg/kg or greater, 350 mg/kg or greater, 400 mg/kg or greater, 450 mg/kg or greater, 500 mg/kg or greater, 550 mg/kg or greater, 600 mg/kg or greater, 700 mg/kg or greater, 750 mg/kg or greater, 700 mg/kg or greater, 850 mg/kg or greater, 900 mg/kg or greater, 950 mg/kg or greater, or 1000 mg/kg or greater) per body weight.

In some embodiments, the PI3K inhibitor (e.g., duvelisib) can be administered to the subject in an amount of 1000 mg/kg grams or less of active ingredient per body weight (e.g., 950 mg/kg or less, 900 mg/kg or less, 850 mg/kg or less, 800 mg/kg or less, 750 mg/kg or less, 700 mg/kg or less, 650 mg/kg or less, 600 mg/kg or less, 550 mg/kg or less, 500 mg/kg or less, 450 mg/kg or less, 400 mg/kg or less, 350 mg/kg or less, 300 mg/kg or less, 250 mg/kg or less, 200 mg/kg or less, 150 mg/kg or less, 125 mg/kg or less, 120 mg/kg or less, 115 mg/kg or less, 110 mg/kg or less, 105 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 8 mg/kg or less, 6 mg/kg or less, or 5 mg/kg or less) per body weight.

The PI3K inhibitor (e.g., duvelisib) can be administered to the subject in an amount ranging from any of the minimum values described above to any of the maximum values described above. In some embodiments, the PI3K inhibitor can be administered to the subject in an amount of from 0.01-1000 mg/kg (e.g., from 0.1-500 mg/kg, from 0.5-100 mg/kg, from 0.1-50 mg/kg, from 1-40 mg/kg, from 5-75 mg/kg, from 5-40 mg/kg, from 5-20 mg/kg, or from 2-20 mg/kg). In some embodiments, the PI3K inhibitor is administered in an amount from 0.1-100 mg/kg.

As described herein, the protein kinase B (Akt) activation inhibitor and second inhibitor (a checkpoint (CHK1) inhibitor, a multitargeted histone deacetylase/human epidermal growth factor receptor (HDAC/Her1/2) inhibitor, a phosphoinositide 3-kinase (PI3K) inhibitor, or a combination thereof) can be used in an amount sufficient to induce a synergistic therapeutic effect while still being pharmaceutically acceptable (i.e., their use in a subject does not result in increased toxicity when compared to the individual application of the pharmaceutical compounds (a) or (b)). The term “synergism” refers to an interaction of two or more factors such that the effect when combined is greater than the predicted effect based on the response to each factor applied eparately.” Synergistic in the pharmaceutical context can mean that the use of (a) and (b) as defined above results in an increased therapeutic effect (such as anticancer) compared to the therapeutic effects that are possible with the use of (a) or (b) alone. In some embodiments, the inhibition to the condition (cancer) caused by the compositions and methods disclosed herein can be evaluated using a scale from 0% to 100%, when compared with the untreated control cancer cells, wherein 0% indicates no inhibition to the undesired cancer cells and 100% indicates complete inhibition of the undesired cancer cells.

In some embodiments, the compositions and methods disclosed herein are synergistic as defined by the Colby equation. See S. R. Colby, Calculating Synergistic and Antagonistic Responses of Herbicide Combinations, WEEDS 15, p. 22 (1967)

E=X+Y−(X*Y)/100

wherein

X=effect in percent using (a) protein kinase B (Akt) activation inhibitor administered in an amount a;

Y=effect in percent using (b) a second inhibitor (a checkpoint (CHK1) inhibitor, a multitargeted histone deacetylase/human epidermal growth factor receptor (HDAC/Her1/2) inhibitor, a phosphoinositide 3-kinase (PI3K) inhibitor, or a combination thereof) administered in an amount b;

E=expected (calculated) effect (in %) of (a)+(b) at application rates a and b.

In Colby's equation, the value E corresponds to the effect (inhibition) that is to be expected if the activity of the individual compounds is additive. If the observed effect is higher than the value E calculated according to the Colby equation, then a synergistic effect is present according to the Colby equation.

In some embodiments, the joint action of the protein kinase B (Akt) activation inhibitor and the second inhibitor can result in enhanced activity against cancer (via synergism), even when administered in amounts below those typically used for the inhibitor to have a therapeutic effect on its own. In some embodiments, the compositions and methods disclosed herein can, based on the individual components, be used at lower application rates to achieve a therapeutic effect comparable to the effect produced by the individual components at normal application rates. In some embodiments, the compositions and methods disclosed herein provide an accelerated action on cancer cells (i.e., they inhibit cancer cells more quickly compared with application of the individual agent).

In some embodiments, the observed inhibitory effect on cancer is at least 1%, at least 2%, at least 3%, at least 4%, at least 5%, at least 10%, at least 15%, at least 20%, at least 25%, at least 27%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, greater than the expected effect (E) calculated according to the Colby equation (e.g., an observed effect of 96% would be 4% greater than a calculated effect (E) of 92%). In some embodiments, for cancer cells, the difference (DO) between 100% and the observed effect is at least 5%, at least 10%, at least 15%, at least 20%, at least 25%, at least 30%, at least 35%, at least 40%, at least 45%, or at least 50% less than the difference (DE) between 100% and the expected effect (E) calculated according to the Colby equation (e.g., an observed effect of 96% would produce a DO of 4%, a calculated effect (E) of 92% would produce a DE of 8%, and DO would be 50% less than or half of DE). In some embodiments, E is greater than X+Y.

In some embodiments, the weight ratio of (a) the protein kinase B (Akt) activation inhibitor in mg/kg to (b) a second inhibitor (a checkpoint (CHK1) inhibitor, a multitargeted histone deacetylase/human epidermal growth factor receptor (HDAC/Her1/2) inhibitor, a phosphoinositide 3-kinase (PI3K) inhibitor, or a combination thereof) in mg/kg, that is sufficient to induce a synergistic therapeutic effect is at least 1:1000 (e.g., at least 1:950, at least 1:900, at least 1:850, at least 1:800, at least 1:750, at least 1:700, at least 1:650, at least 1:600, at least 1:550, at least 1:500, at least 1:450, at least 1:400, at least 1:350, at least 1:300, at least 1:250, at least 1:200, at least 1:150, at least 1:100, at least 1:90, at least 1:80, at least 1:70, at least 1:60, at least 1:50, at least 1:45, at least 1:40, at least 1:35, at least 1:30, at least 1:25, at least 1:20, at least 1:18, at least 1:16, at least 1:15, at least 1:14, at least 1:12, at least 1:10, at least 1:9, at least 1:8, at least 1:7, at least 1:6, at least 1:5, at least 1:4, at least 1:3, at least 1:2, at least 1:1, at least 2:1, at least 3:1, at least 4:1, at least 5:1, at least 6:1, at least 7:1, at least 8:1, at least 9:1, at least 10:1, at least 20:1, at least 30:1, at least 40:1, at least 50:1, at least 100:1, at least 150:1, at least 200:1, at least 250:1, at least 300:1, or at least 350:1). In some embodiments, the weight ratio of (a) the protein kinase B (Akt) activation inhibitor in mg/kg to (b) a second inhibitor (a checkpoint (CHK1) inhibitor, a multitargeted histone deacetylase/human epidermal growth factor receptor (HDAC/Her1/2) inhibitor, a phosphoinositide 3-kinase (PI3K) inhibitor, or a combination thereof) in mg/kg, that is sufficient to induce a synergistic therapeutic effect is less than 400:1 (e.g., less than 350:1, less than 300:1, less than 250:1, less than 200:1, less than 150:1, less than 100:1, less than 90:1, less than 80:1, less than 70:1, less than 60:1, less than 50:1, less than 45:1, less than 40:1, less than 35:1, less than 30:1, less than 25:1, less than 20:1, less than 18:1, less than 17:1, less than 16:1, less than 15:1, less than 10:1, less than 9:1, less than 8:1, less than 7:1, less than 6:1, less than 5:1, less than 4:1, less than 3:1, less than 2:1, less than 1:1, less than 1:2, less than 1:3, less than 1:4, less than 1:5, less than 1:6, less than 1:7, less than 1:8, less than 1:9, or less than 1:10, less than 1:15, less than 1:20, less than 1:30, less than 1:40, less than 1:50, less than 1:60, less than 1:70, less than 1:80, less than 1:90, less than 1:100, less than 1:150, less than 1:200, less than 1:250, less than 1:300, less than 1:350, less than 1:400, less than 1:450, less than 1:500, less than 1:600, less than 1:700, less than 1:800, less than 1:900, or less than 1:950).

The weight ratio of (a) the protein kinase B (Akt) activation inhibitor in mg/kg to (b) a second inhibitor (a checkpoint (CHK1) inhibitor, a multitargeted histone deacetylase/human epidermal growth factor receptor (HDAC/Her1/2) inhibitor, a phosphoinositide 3-kinase (PI3K) inhibitor, or a combination thereof) in mg/kg that is sufficient to induce a synergistic therapeutic effect can range from any of the minimum ratios described above to any of the maximum values described above. In some embodiments, the weight ratio of (a) the protein kinase B (Akt) activation inhibitor in mg/kg to (b) a second inhibitor (a checkpoint (CHK1) inhibitor, a multitargeted histone deacetylase/human epidermal growth factor receptor (HDAC/Her1/2) inhibitor, a phosphoinositide 3-kinase (PI3K) inhibitor, or a combination thereof) in mg/kg, that is sufficient to induce a synergistic therapeutic effect is from 1:1000 to 400:1 (e.g., 1:800 to 400:1, 1:900 to 350:1, 1:800 to 300:1, 1:500 to 200:1, 1:200 to 100:1, 1:60 to 50:1, 1:50 to 45:1, 1:40 to 20:1, 1:10 to 50:1, 1:20 to 5:1, 1:20 to 1:1, 1:16 to 2.5:1, 1:15 to 10:1, 1:12 to 2.5:1, 1:10 to 10:1, 1:8 to 5:1, 1:6.4 to 5:1, 1:5 to 2:1, 1:5 to 1:1, 1:1 to 1:5, 1:1 to 1:20, 1:6.4 to 5:4 or 1:2.5 to 6.5:1).

In some embodiments, the active ingredients consist of (a) the protein kinase B (Akt) activation inhibitor and (b) the second inhibitor, i.e., the composition does not include a therapeutic active ingredient in addition to (a) and (b).

The present disclosure also relates to formulations of the compositions and methods disclosed herein. In some embodiments, the formulation can be in the form of a single package formulation including both (a) the protein kinase B (Akt) activation inhibitor and (b) a second inhibitor (a checkpoint inhibitor, a multitargeted histone deacetylase/human epidermal growth factor receptor inhibitor, a phosphoinositide 3-kinase inhibitor, or a combination thereof). In some embodiments, the formulation can be in the form of a single package formulation including both (a) and (b) and further including at least one additive.

In some embodiments, the formulation can be in the form of a two-package formulation, wherein one package contains compound (a) and optionally at least one additive while the other package contains compound (b) and optionally at least one additive. In some embodiments of the two-package formulation, the formulation including compound (a) and optionally at least one additive and the formulation including compound (b) and optionally at least one additive are mixed before administration and then applied simultaneously. In some embodiments, the formulation including compound (a) and the formulation including compound (b) are not mixed but are administered sequentially (in succession), for example, immediately or within 1 hour, within 2 hours, within 4 hours, within 8 hours, within 16 hours, within 24 hours, within 2 days, or within 3 days, of each other.

The compositions and methods disclosed herein can also be administered with an additive. In some embodiments, the additive can be an additional therapeutic agent.

### Methods

Methods of using the disclosed compositions for treating, preventing, reducing, and/or inhibiting a cancer comprising administering the same to a subject are also disclosed. The methods can further comprise administering a third compound or composition, such as, for example, anticancer agents or anti-inflammatory agents. Additionally, the method can further comprise administering an effective amount of ionizing radiation to the subject.

Methods of killing a tumor cell are also provided herein. The methods comprise contacting a tumor cell with an effective amount of a r composition as disclosed herein. The methods can further include administering a third compound or composition (e.g., an anticancer agent or an anti-inflammatory agent) or administering an effective amount of ionizing radiation to the subject.

Also provided herein are methods of radiotherapy of tumors, comprising contacting the tumor with an effective amount of a composition as disclosed herein and irradiating the tumor with an effective amount of ionizing radiation.

Also disclosed are methods for treating oncological disorders in a patient. In one embodiment, a synergistic effective amount of one or more compositions disclosed herein is administered to a patient having an oncological disorder and who is in need of treatment thereof. The disclosed methods can optionally include identifying a patient who is or can be in need of treatment of an oncological disorder. The patient can be a human or other mammal, such as a primate (monkey, chimpanzee, ape, etc.), dog, cat, cow, pig, or horse, or other animals having an oncological disorder. Oncological disorders include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

In some aspect, disclosed are methods for treating a tumor or tumor metastases in a subject by the administration to the subject a combination of at least one composition as disclosed herein and at least one cancer immunotherapeutic agent. The disclosed compositions can be administered alone or in combination with a cancer immunotherapeutic agent. The subject can receive the therapeutic compositions prior to, during or after surgical intervention to remove all or part of a tumor. Administration may be accomplished via direct immersion; systemic or localized intravenous (i.v.), intraperitoneal (i.p.), subcutaneous (s.c.), intramuscular (i.m.), or direct injection into a tumor mass; and/or by oral administration of the appropriate formulations.

In specific examples, the type of cancer is leukemia.

Administration

The disclosed compounds in the compositions can be administered either sequentially or simultaneously in separate or combined pharmaceutical formulations. The dose of each compound can be either the same as or differ from that when the compound is used alone. Appropriate doses will be readily appreciated by those skilled in the art.

The term “administration” and variants thereof (e.g., “administering” a composition) in reference to a compound of the invention means introducing the composition or a prodrug of the composition into the system of the animal in need of treatment. Administration is understood to include concurrent and sequential introduction of the compound or prodrug thereof and other agents in the compositions.

In vivo application of the disclosed compositions can be accomplished by any suitable method and technique presently or prospectively known to those skilled in the art. For example, the disclosed compositions can be formulated in a physiologically- or pharmaceutically-acceptable form and administered by any suitable route known in the art including, for example, oral, nasal, rectal, topical, and parenteral routes of administration.

As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compositions disclosed herein can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compositions disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compositions disclosed herein can be delivered to a cell either through direct contact with the cell or via a carrier means. Carrier means for delivering compositions to cells are known in the art and include, for example, encapsulating the composition in a liposome moiety. Another means for delivery of compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 2002/0035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compositions disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as Adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Many tumors and cancers have viral genome present in the tumor or cancer cells. For example, Epstein-Barr Virus (EBV) is associated with a number of mammalian malignancies. The compositions disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease. For example, the compounds can be used with mutant herpes simplex virus in the treatment of non-small cell lung cancer (Toyoizumi, et al., “Combined therapy with chemotherapeutic agents and herpes simplex virus type IICP34.5 mutant (HSV-1716) in human non-small cell lung cancer,” Human Gene Therapy, 1999, 10(18):17).

Therapeutic application of compositions can be accomplished by any suitable therapeutic method and technique presently or prospectively known to those skilled in the art. Further, compounds and compositions disclosed herein have use as starting materials or intermediates for the preparation of other useful compounds and compositions.

Compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compositions and agents disclosed herein can be applied in as a liquid or solid. However, it will generally be desirable to administer them topically to the skin as compositions, in combination with a dermatologically acceptable carrier, which can be a solid or a liquid. Compositions disclosed herein can be applied topically to a subject's skin to reduce the size (and can include complete removal) of malignant or benign growths, or to treat an infection site. Compositions and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of oncological disorders, compounds and agents and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as Adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³² etc.), and toxins of bacterial, fungal, plant, or animal origin (e.g., ricin, botulinum toxin, anthrax toxin, aflatoxin, jellyfish venoms (e.g., box jellyfish), etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

Kits

Kits for practicing the methods of the invention are further provided. By “kit” is intended any manufacture (e.g., a package or a container) comprising anyone of the compositions described herein. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

### Example 1

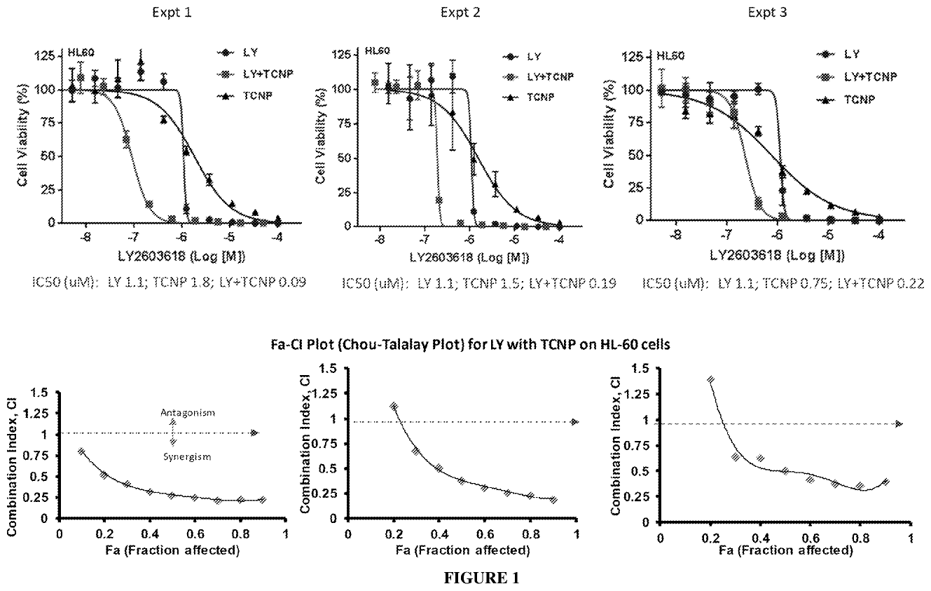

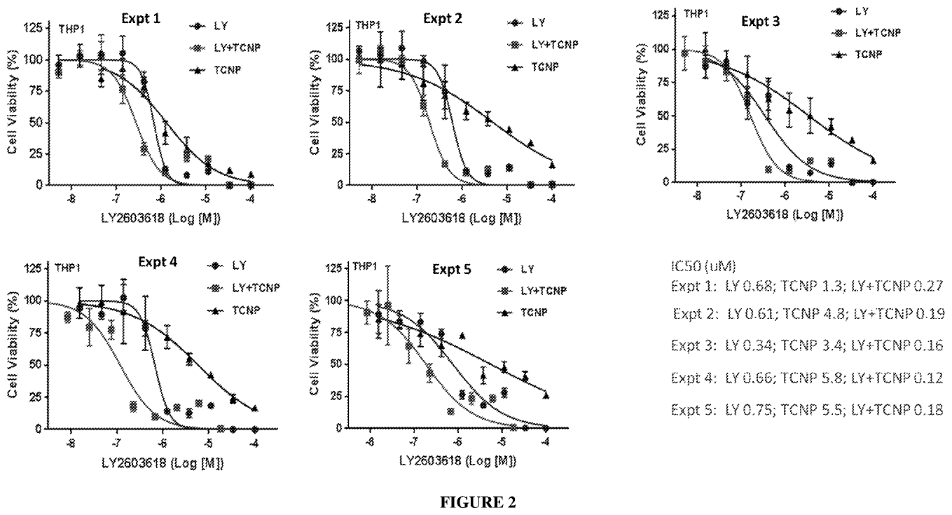

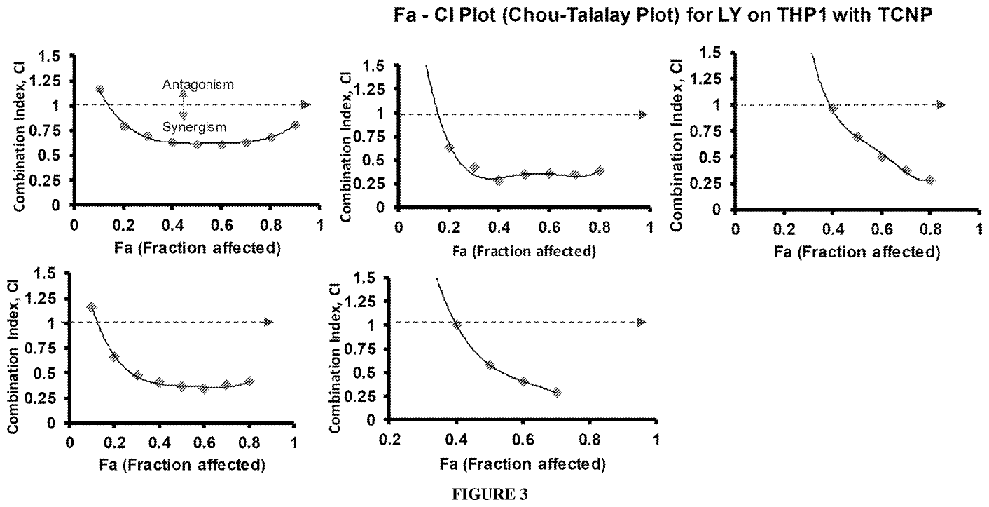

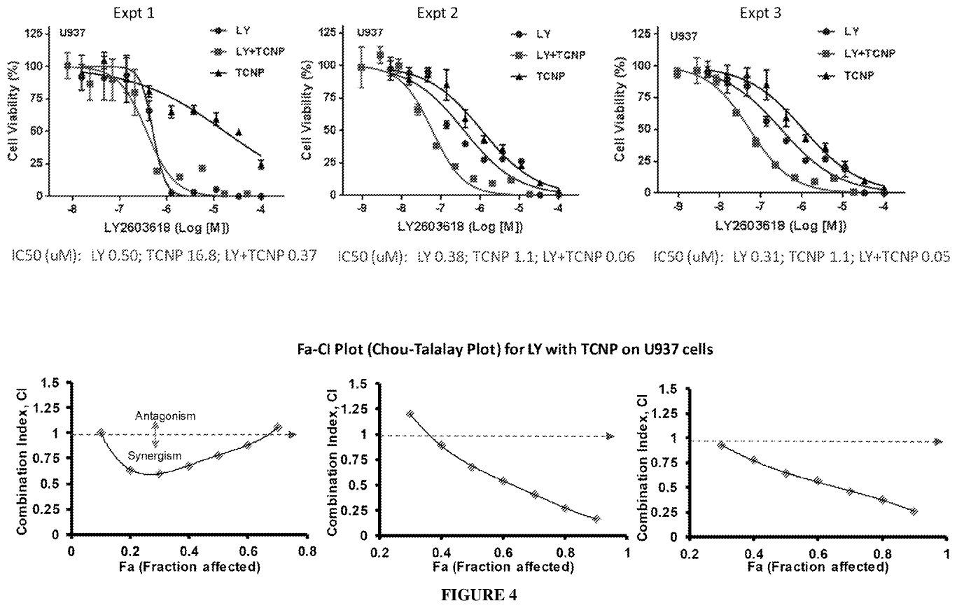

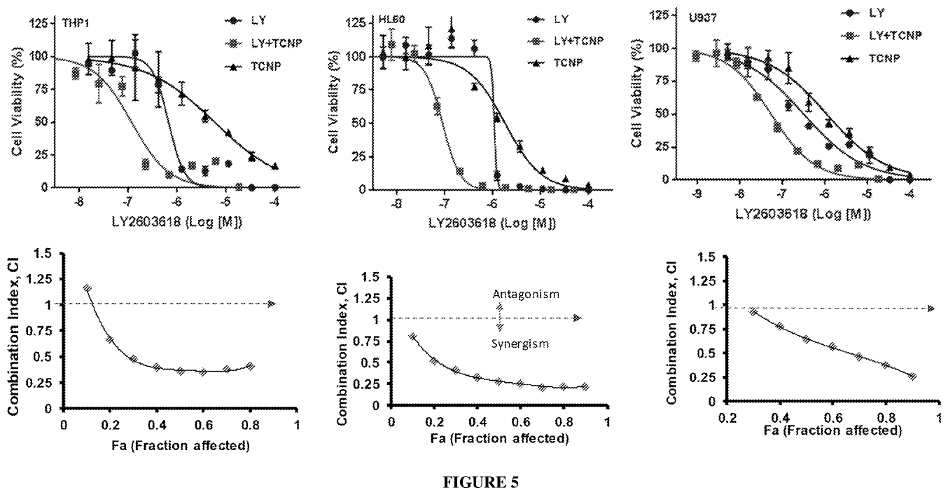

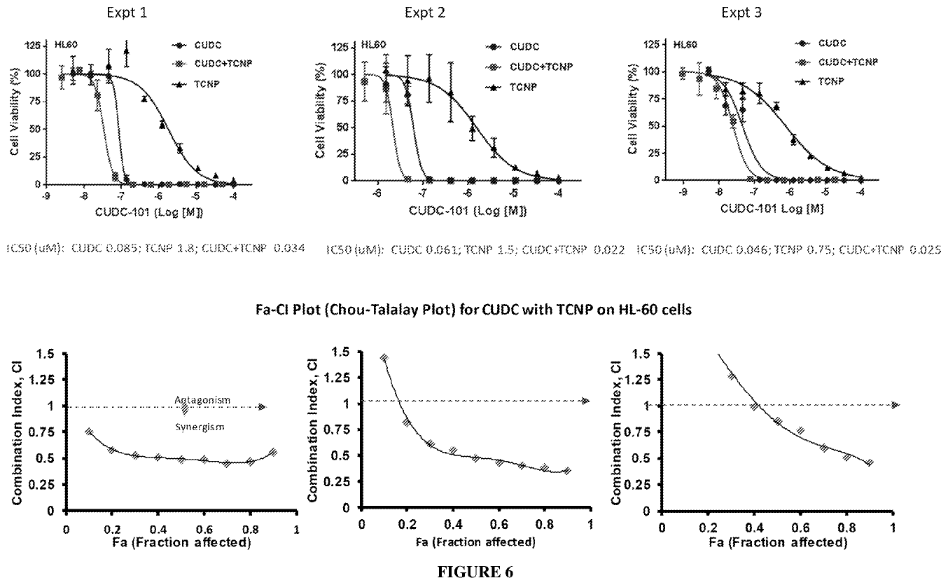

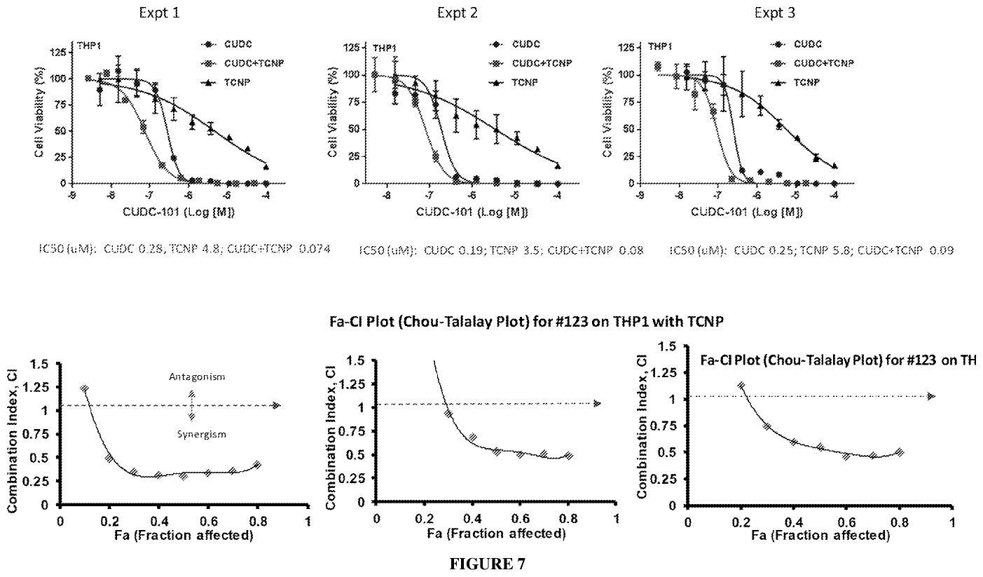

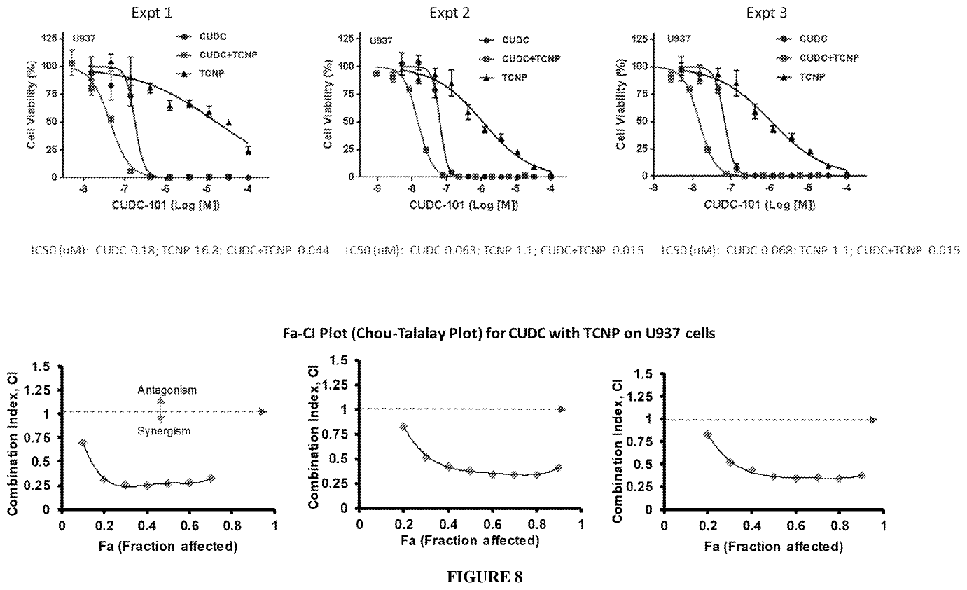

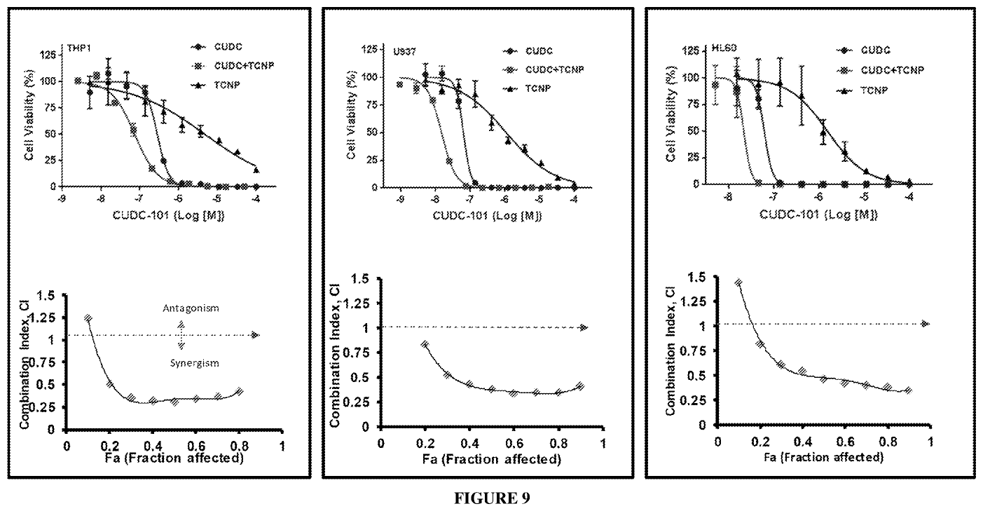

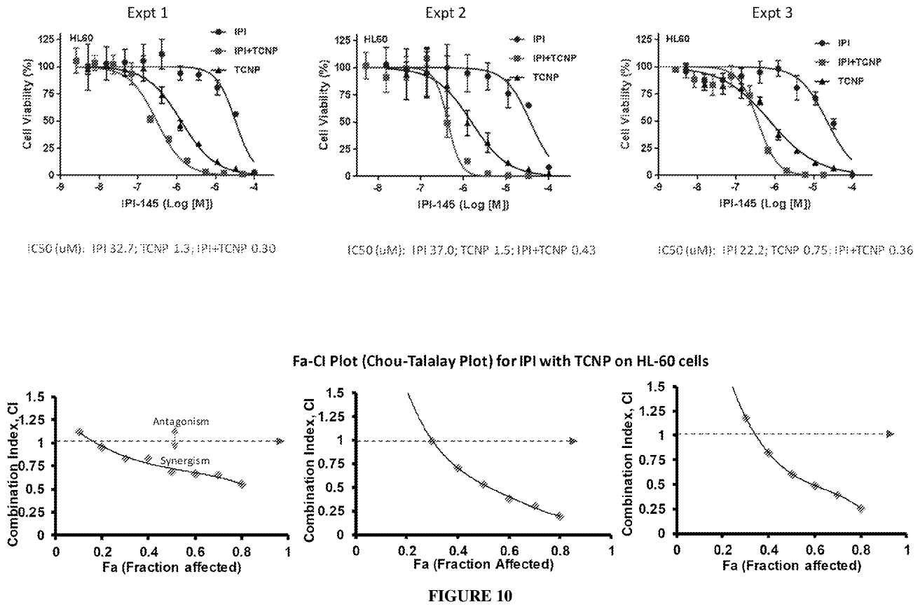

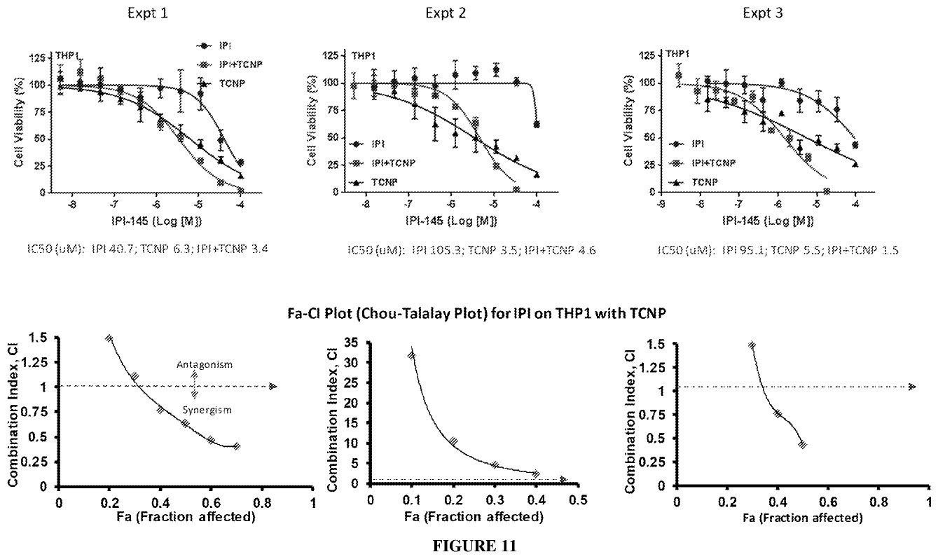

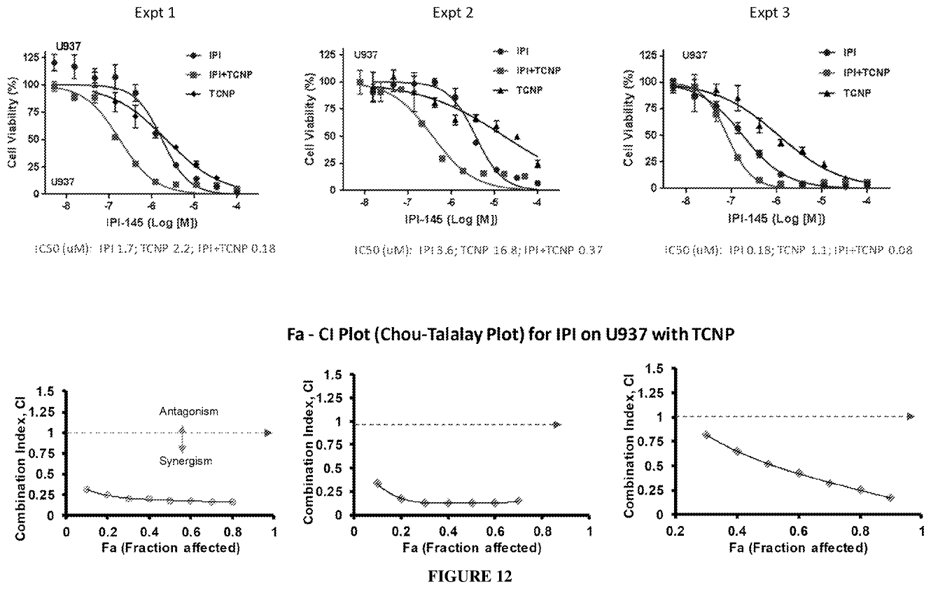

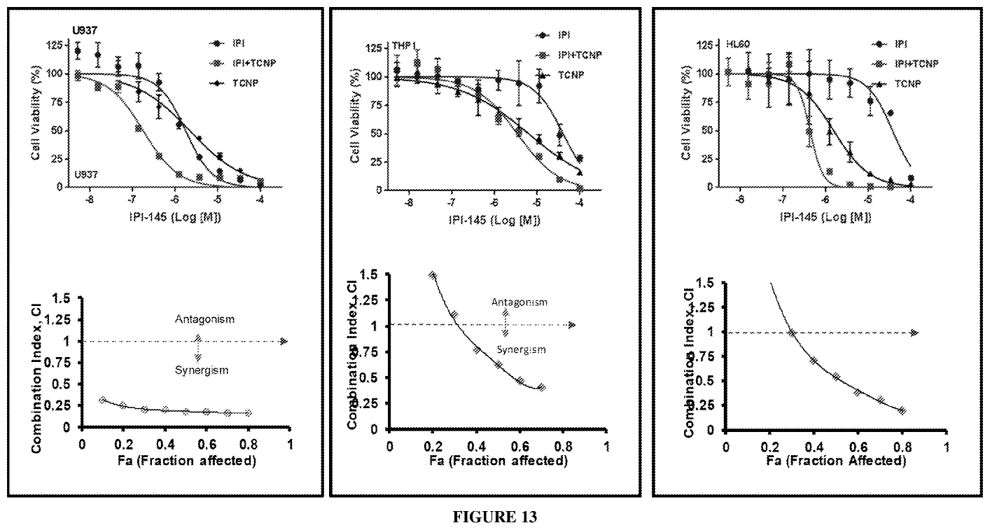

FIGS. 1-13 exemplifies clinical drugs that sensitize leukemia cells to the Akt Activation Inhibitor TCNP. The following sensitizers to TCNP were identified in THP1 leukemia cells:

1. The CHK1 inhibitor LY2603618

2. The HDAC/Her1/2 inhibitor CUDC-101

3. The PI3K inhibitor IPI-145.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A pharmaceutical composition comprising a therapeutically effective synergistic amount of a protein kinase B (Akt) activation inhibitor and a second inhibitor selected from a checkpoint (CHK1) inhibitor, a multitargeted histone deacetylase/human epidermal growth factor receptor (HDAC/Her1/2) inhibitor, a phosphoinositide 3-kinase (PI3K) inhibitor, or a combination thereof.

2. (canceled)

3. The composition of claim 1, wherein the Akt activation inhibitor comprises triciribine phosphate (TCN-P).

4. The composition of claim 1, wherein the composition comprises the Akt activation inhibitor and the CHK1 inhibitor in a therapeutically effective synergistic amount.

5. (canceled)

6. The composition of claim 1, wherein the CHK1 inhibitor comprises rabusertib (LY2603618).

7. The composition of claim 1, wherein the weight ratio of Akt activation inhibitor to CHK1 inhibitor is from 1:50 to 50:1.

8. The composition of claim 1, wherein the active ingredients in the composition consist of Akt activation inhibitor and CHK1 inhibitor.

9. (canceled)

10. The composition of claim 1, wherein the HDAC/Her1/2 inhibitor comprises CUDC-101.

11. The composition of claim 1, wherein the weight ratio of Akt activation inhibitor to HDAC/Her1/2 inhibitor is from 1:50 to 50:1.

12. The composition of claim 1, wherein the active ingredients in the composition consist of Akt activation inhibitor and HDAC/Her1/2 inhibitor.

13. (canceled)

14. (canceled)

15. The composition of claim 1, wherein the PI3K inhibitor comprises duvelisib (IPI-145).

16. The composition of claim 1, wherein the weight ratio of Akt activation inhibitor to PI3K inhibitor is from 1:50 to 50:1.

17. The composition of claim 1, wherein the active ingredients in the composition consist of Akt activation inhibitor and PI3K inhibitor.

18. The composition of claim 1, further comprising an additional therapeutic active ingredient.

19. A method for treating a cancer in a subject, comprising administering to the subject a composition of claim 1.

20. The method of claim 19, wherein the cancer is selected from breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, or plasma cell neoplasm (myeloma).

21. The method of claim 19, wherein the cancer is leukemia.

22. (canceled)

23. The method of claim 19, wherein Akt activation inhibitor is administered in an amount of 0.1 mg/kg body weight or greater.

24. The method of claim 19, wherein CHK1 inhibitor is administered in an amount of 0.1 mg/kg body weight or greater.

25. The method of claim 19, wherein HDAC/Her1/2 inhibitor is administered in an amount of 0.1 mg/kg body weight or greater.

26. The method of claim 19, wherein PI3K inhibitor is administered in an amount of 0.1 mg/kg body weight or greater.
